(19) United States
(12) Patent Application Publication
(10) Pub. No.: US 2011/0262319 A1
(43) Pub. Date: Oct. 27, 2011

(54) STERLIZING SYSTEM, AND LOADING/UNLOADING DEVICE THEREFOR

(75) Inventor: Staffan Svensson, Halmstad (SE)

(73) Assignee: REMEDA AB, Halmstad (SE)

(21) Appl. No.: 13/090,870

(22) Filed: Apr. 20, 2011

Related U.S. Application Data
(60) Provisional application No. 61/328,028, filed on Apr. 26, 2010.

(30) Foreign Application Priority Data
Apr. 26, 2010 (SE) 1050411-6

(51) Int. Cl. A61L 2/07; B65G 65/00

(52) U.S. Cl. 422/298; 414/222.04; 414/222.02

(57) ABSTRACT

An loading/unloading device having at least two loading arms is provided. One loading surface is used for receiving a load to be sterilized and transferring the load to a sterilizing device. One unloading surface is used for receiving a sterilized load from a sterilizing device and for transporting the sterilized load away from the sterilizing device. The loading/unloading device is able to be laterally moved such that the loading surface or the unloading surface at least may be aligned to the opening of the sterilizing device.

This application claims benefit of Serial No. 1050411-6, filed 26 Apr. 2010 in Sweden and Ser. No. 61/328,028, filed 26 Apr. 2010 in the United States and which applications are incorporated herein by reference. To the extent appropriate, a claim of priority is made to each of the above disclosed applications.

### FIELD OF THE INVENTION

This invention pertains in general to the field of sterilizing equipment, and more particularly to a sterilizing system and a loading/unloading device therefore.

### BACKGROUND OF THE INVENTION

Sterilizing equipments, such as autoclaves are widely used in many sciences, such as medicine, dentistry, veterinary science etc. The basic concept of an autoclave is to sterilize a load, such as glassware, medical waste, or utensils, etc. by subjecting them to high pressure steam and superheated water at 121° C. or more. A key issue is to ensure that all of the air trapped in the load is removed, since hot air is poor at achieving sterility.

One commonly known type of autoclave is the front-loaded autoclave. Here, the loading and unloading of the load is made through an opening on the front of the autoclave. The loading and unloading of the load may be performed automatically by means of a single conveyor belt.

However, such a solution suffers from poor throughput, since the sterilized load has to be moved away by means of the conveyor belt driven in a backward direction before a new unsterilized load may be loaded into the autoclave by means of the conveyor belt driven in a forward direction.

Hence, an improved sterilizing system would be advantageous.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a device, system, control unit, and methods according to the appended patent claims.

According to an aspect a device for transferring a load into or from a sterilizing device is provided. The device comprises a loading surface for transporting a load to be sterilized into an opening of the sterilizing device. The device further comprises an unloading surface for transporting a sterilized load from the opening of the sterilizing device. Furthermore, the device comprises a means for laterally moving the device in relation to the opening of the sterilizing device to align the loading surface or unloading surface with the opening of the sterilizing device.

In an aspect a control unit is provided. The control unit is configured receive signals from a signal unit and control at least the alignment of the loading surface and unloading surface to the sterilizing device.

According to a further aspect a system comprising the device and a sterilizing device is provided.

According to yet another aspect a method for loading a load into a sterilizing device is provided. The method comprises receiving a load to be sterilized onto a loading surface of a device, the device further comprising an unloading surface. Moreover the method comprises aligning the loading surface with an opening of the sterilizing device, and loading the load to be sterilized onto the sterilizing device.

In a further aspect a method for unloading a sterilized load from a sterilizing device is provided. The method comprises aligning an unloading surface of a device further comprising a loading surface with an opening of the sterilizing device. The method further comprises unloading the sterilized load from the sterilizing device to the unloading surface of the device.

The device of the present invention is safe, and allows for reduced process times thereby increasing the throughput efficiency for sterilizing devices having one opening for both loading and unloading of loads.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1a is an illustration showing a loading/unloading device according to an embodiment;

FIG. 1b is a side view of the loading/unloading device of FIG. 1a;

FIG. 1c is a top view of the loading/unloading device of FIG. 1a;

FIG. 2 is an illustration showing a loading/unloading device according to an embodiment, being aligned in a loading position in relation to a sterilizing device;

FIG. 3 is an illustration showing a loading/unloading device according to an embodiment, being aligned in an unloading position in relation to a sterilizing device;

FIG. 4 is an illustration showing a sterilizing system according to an embodiment;

FIG. 5 is an illustration showing a sterilizing system according to an embodiment; and

FIGS. 6 to 15 each illustrate the sterilizing system according to an embodiment in different phases of loading and unloading.

### DESCRIPTION OF EMBODIMENTS

Several embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

The following description focuses on embodiments applicable to a sterilizing system and in particular to a loading/unloading device for a front loaded sterilizing device.

An idea of the invention is to provide a loading/unloading device having at least two surfaces. One surface, called loading surface is used for receiving a load to be sterilized and transferring the load to a sterilizing device. The other surface, called unloading surface is used for receiving a sterilized load from a sterilizing device and for transporting the sterilized load away from the sterilizing device. The loading/unloading device is able to be laterally moved such that the loading surface or the unloading surface at least may be aligned to the opening of the sterilizing device.

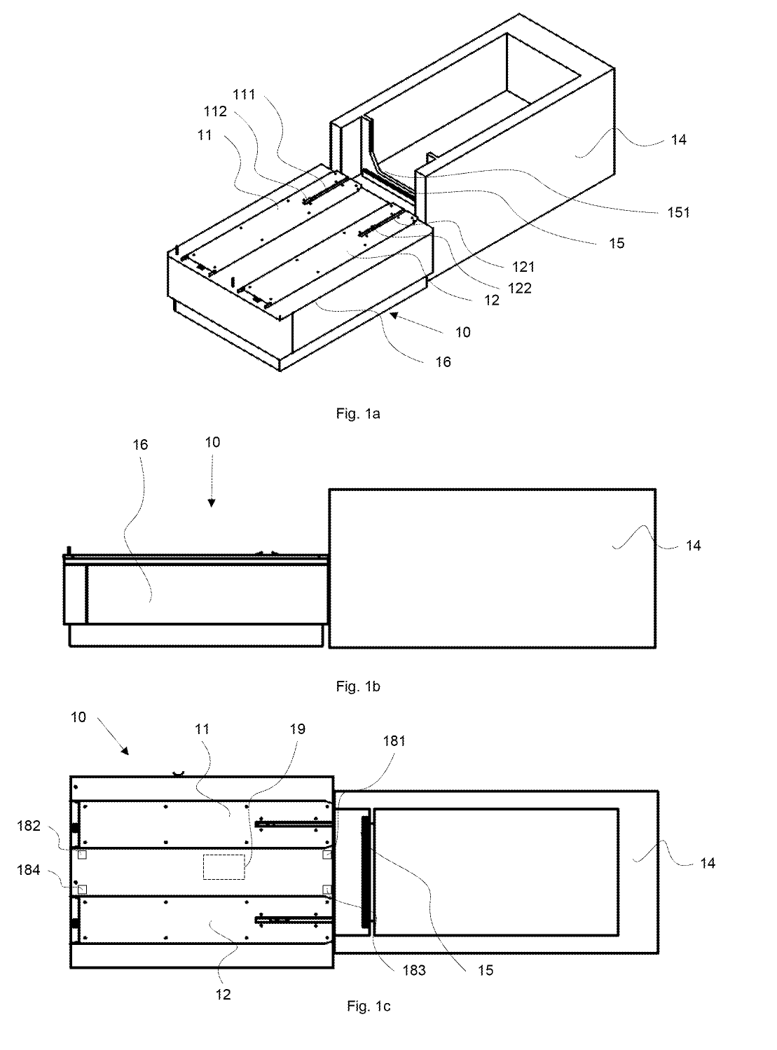

In an embodiment, according to FIG. 1a, a loading/unloading device 10 for a sterilizing device 14 is provided. The loading/unloading device 10 comprises a housing 16 in which a loading surface 11 for supporting a load (not shown) to be loaded into the sterilizing device 14 via a front door opening 151 is provided. An unloading surface 12 for supporting a sterilized load received from the sterilizing device 14 via the front door opening 151 is also provided in the housing 16. The loading surface 11 comprises means for receiving a load, e.g. a loading arm 111 which is slidably arranged in the loading surface. The loading arm 111 comprises an engaging loading means 112, such as a hook, for engaging the loading arm 111 to the load (not shown). When engaged the loading arm 111 is configured to move the engaged load into the sterilizing device 14.

The unloading surface 12 may correspondingly comprise an unloading arm 121 having an engaging unloading means 122, such as a hook, for engaging the loading arm 111 to a sterilized load (not shown) located in the sterilizing device 14. When engaged the unloading arm 121 is configured to move the engaged sterilized load from the sterilizing device 14 and onto the unloading surface 12.

The loading arm 111 and/or the unloading arm 121 with corresponding engaging loading and/or unloading means may be controlled by means of at least one motor (not shown) which is preferably comprised within the loading/unloading device 10.

The loading/unloading device further comprises a means for laterally moving the device in relation to the opening 151 of the sterilizing device to align the loading surface 11 or unloading surface 12 with the opening 151 of the sterilizing device.

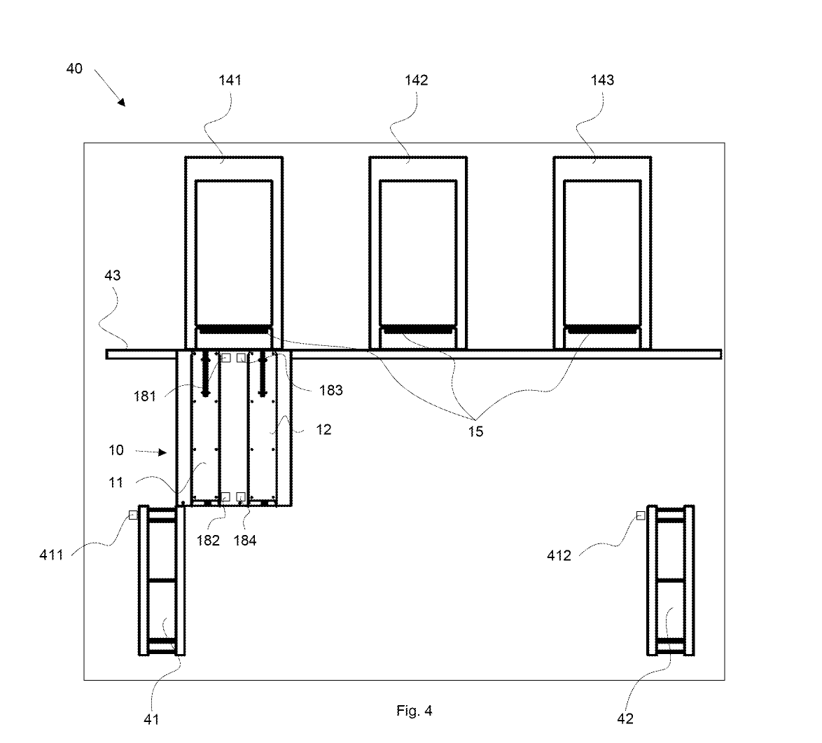

In an embodiment, the means for laterally moving the loading/unloading device comprises a rail 43, see FIG. 4, being arranged transversely in respect to the first and/or second loading arm, along which rail the housing 16 may slide by means of a recess corresponding to the rail provided in the bottom portion of the housing. The sides of the recess may e.g. be provided with a pair of wheels, preferably made of rubber and being connected to a positioning motor (not shown), thereby enabling the housing to move laterally along the rail to a desired position in a controlled manner. The loading/unloading device 10 may be guide steered sideways between the loading and unloading positions by means of the positioning motor driving the pair of wheels.

FIG. 1b illustrates the loading/unloading device of the preferred embodiment seen from a side view.

FIG. 1c illustrates the loading/unloading device of the preferred embodiment seen from a top view.

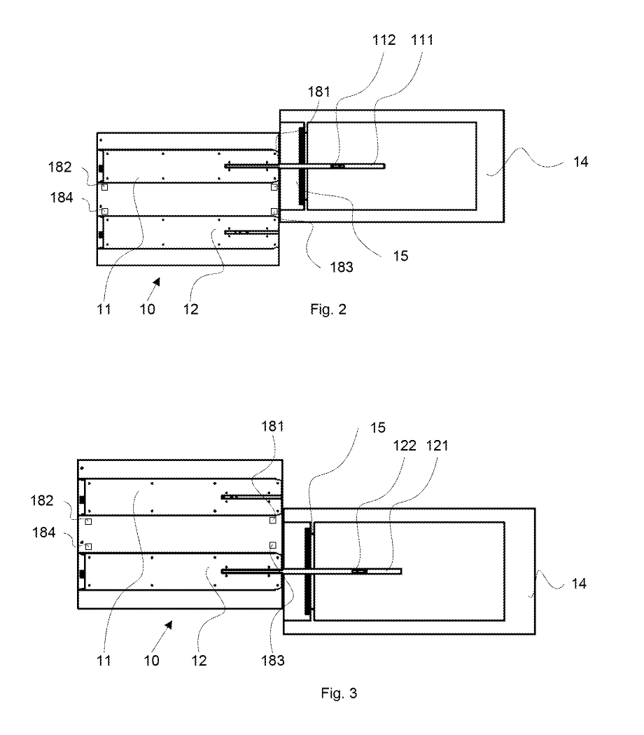

When a load is to enter the sterilizing device 14 the housing 16 is laterally positioned such that the loading surface is aligned with the door opening 151 of the sterilizing device. This lateral position is referred to as loading position and is illustrated in FIG. 2. After the housing is positioned in the loading position the load may enter the sterilizing device 14 by engaging the loading means 112 to the load (not shown) followed by driving the loading arm 111 in a forward manner until fully extended, as shown in FIG. 2. When the load has fully entered the sterilizing device 14, the engaging loading means 121 is disengaged and the loading arm 111 is retracted towards to its original position, whereby the opening 15 of the sterilizing device 14 is closed and the sterilization procedure begins.

When a load has entered the sterilizing device 14 the housing is laterally positioned such that the unloading surface 12 is aligned with the door opening 151. This lateral position is referred to as unloading position and is illustrated in FIG. 3. When a load has been sterilized in the sterilizing device 14, and the housing is positioned in the unloading position, the sterilized load may be transferred from the sterilizing device 14 and onto the unloading surface 12. Once the door 15 of the sterilizing device 14 is opened the sterilized load is transferred from the sterilizing device 14 by extending the unloading arm 121, as shown in FIG. 3 and engaging the engaging unloading means 122 to the sterilized load (not shown) followed by retracting the unloading arm to pull the sterilized load from the sterilizing device 14 towards the unloading surface 12.

In an embodiment, according to FIG. 4, a sterilizing system 40 is provided. The sterilizing system comprises a loading/unloading device 10 and at least one sterilizing device 15, 141, 142, 143. The rail 43 along which the housing of the loading/unloading device may be moved is illustrated in FIG. 4. The length of the rail is adapted such that the loading/unloading device 10 may be laterally moved in relation to each of the sterilizing device comprised in the sterilizing system. For example, in FIG. 4, the rail length is selected such that the loading/unloading device may be laterally moved along the rail in order to be able to be positioned in the loading position and unloading position of each sterilizing device.

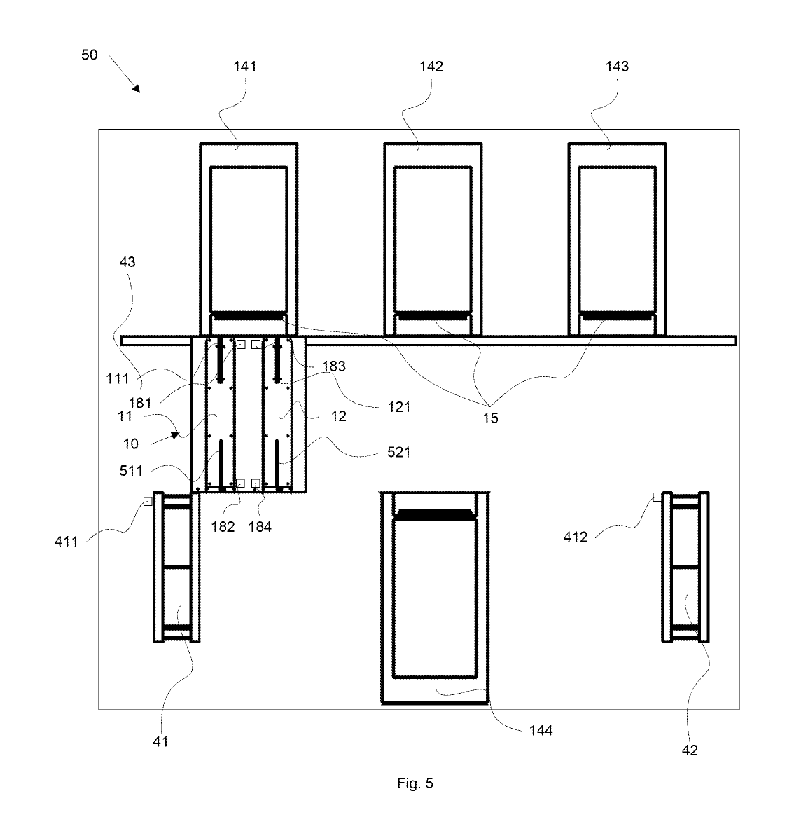

The system may further comprise a loading conveyor 41 which is capable of providing loads to be sterilized onto the loading surface 11 on the loading/unloading device 10, e.g. by means of a second loading arm 511 arranged on the loading surface 11, as shown in FIG. 5, or on the loading conveyor 41, as shown in FIG. 7. In order for a load to be transferred to the loading/unloading device 10 the housing 16 of the loading/unloading device 10 is laterally positioned by means of the rail to a second loading position, in which the loading surface 11 is aligned with the loading conveyor 41. Hence, the length of the rail is adapted to enable the housing 16 to obtain the second loading position, as is illustrated in FIG. 4.

The system may further comprise an unloading conveyor 42 which is capable of collecting sterilized loads from the unloading surface 12 on the loading/unloading device 10, e.g. by means of a second unloading arm 521 arranged on the unloading surface 12 as shown in FIG. 5, or on the unloading conveyor, as shown in FIG. 13. In order for a load to be transferred to the unloading conveyor 42 the housing 16 of the loading/unloading device 10 is laterally positioned by means of the rail to a second unloading position, in which the unloading surface 12 is aligned with the unloading conveyor 42. Hence, the length of the rail is adapted to enable the housing 16 to obtain the second unloading position, as is illustrated in FIG. 4.

In an embodiment the load to be sterilized is located on a tray or shelf stand, whereby the tray or shelf stand including the load is transferred into the sterilizing device by means of the loading arm 111 or transferred from the sterilizing device by means of the unloading arm 121.

Signal Units

In an embodiment the sterilizing device 14 comprises a first signal unit. The first signal unit is configured to send a first signal to a control unit 19 when the sterilizing device is ready to receive a load. The first signal is received by the control unit 19, which is optionally incorporated in the loading/unloading device 10 as illustrated in FIG. 1c, whereby the control unit 19 is configured to control the positioning motor such that the housing of the loading/unloading device 10 is moved to the loading position. The first signal unit may also be configured to send a second signal to the control unit 19 when the door 15 of the sterilizing device 14 is opened, and thereby that the sterilizing device 14 is ready for loading the load.

In an embodiment, the loading/unloading device 10 comprises a second signal unit. The second signal unit may be configured to send a third signal to the control unit 19 when a load originally located on the loading surface 11 has been transferred into the sterilizing device 14. The signal may be triggered by a first sensor 181, such as a motion detector, as illustrated in FIG. 1c, situated at a first end of the loading surface 11, located in close proximity to the sterilizing device 14. Once the load has passed the first sensor 181, which gives rise to the third signal, the load is fully transferred into the sterilizing device 14. The control unit 19 is configured to initiate the sterilization procedure of the sterilizing device 14 upon receipt of the third signal, whereby the door 15 of the sterilizing device 14 is closed.

By receipt of the third signal the control unit 19 may also be configured to control the positioning motor such that the housing 16 is moved into the unloading position, in the case that no new load is ready to be transferred onto the loading surface 11.

Before the housing 16 is moved into the unloading position, alternatively, a new load to be sterilized may enter the loading surface 11, e.g. from the loading conveyor. A second sensor 182 situated at the front end of the loading/unloading device 10 may be used to detect when a new load has entered the loading surface 11, which triggers a fourth signal, in the second signal unit, which is sent to the control unit 19, whereby the control unit 19 is configured to control the positioning motor such that the housing 16 is moved into the unloading position in order for the unloading surface 12 to receive load which is located in the sterilizing device 14.

According to an embodiment, each end of the loading surface and unloading surface may be provided with a blocking sensor detecting whether the corresponding end of the loading surface or unloading surface is blocked by a load/sterilized load. Each blocking sensor, triggers a signal which is sent to the control unit, whereby the control unit limits the ability of moving the loading/unloading device, until the load is safely transported onto or off the loading/unloading device. This is advantageous for safety reasons.

The loading conveyor 41 may comprise a fifth sensor 411 which by detection of a load to be sterilized triggers a loading signal which is sent to the control unit. Hence, by detection of the loading signal the control unit 19 is configured to control the positioning motor such that the housing 16 is moved into the second loading position.

When the sterilization procedure is complete, the first signal unit sends a fifth signal to the control unit 19 which is configured to open the door 15 of the sterilizing device 14, whereby the sterilized load may be transferred onto the unloading surface 12 by means of the unloading arm 121. A third sensor 183 situated at a first end of the unloading surface 12, located in close proximity to the sterilizing device 14, detects when the sterilized load has been transferred to the unloading surface 12, whereby a sixth signal is sent from the second signal unit to the control unit 19. By receipt of the sixth signal the control unit is configured to control the positioning motor such that the housing is transferred to the second unloading position, thereby aligning the unloading surface 12 with the unloading conveyor 42.

The unloading conveyor 42 may comprise a sixth sensor 412 which by detection of a sterilized load triggers an unloading signal which is sent to the control unit. Hence, by detection of the unloading signal a confirmation that the sterilized load has left the loading/unloading device 10 is registered in the control unit 19.

If a load is present on the loading surface 11, which is indicated by the fourth signal, and the sterilizing device 14 is ready to receive a new load to be sterilized, which is indicated by the first signal, the control unit may control the positioning motor such that the housing is moved to the loading position, and the load is transferred into the sterilizing device 14 in the same manner as earlier described.

The first 181, second 182, third 183, fourth 184, fifth 411, and sixth 412 sensors may be connected to the second signal unit, from which a corresponding signal may be sent to the control device 19.

In an embodiment the functionality of the first signal unit and second signal unit may be comprised in a single signal unit.

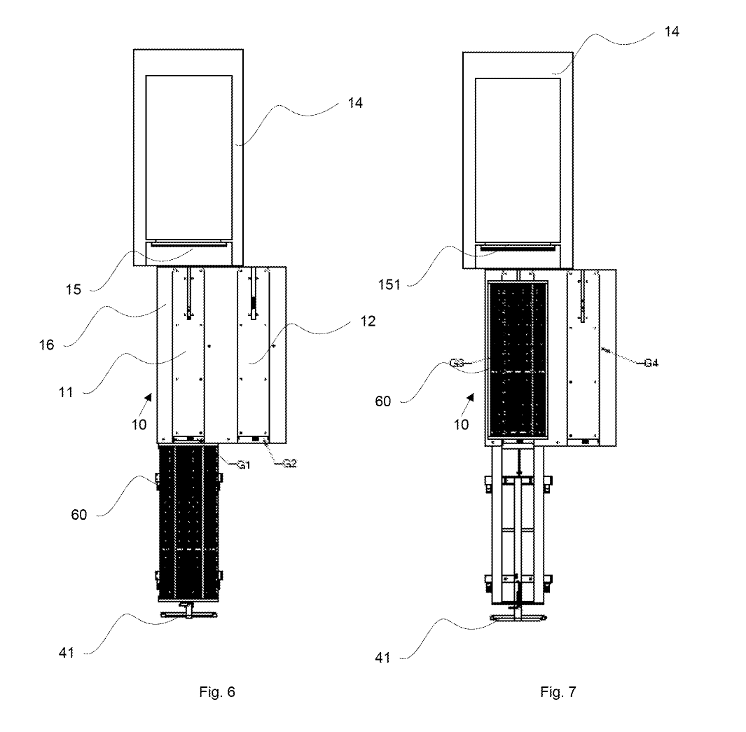

FIGS. 6 to 15 are showing different phases of loading/unloading according to some embodiments. In FIG. 6, the loading/unloading device 10 is located in front of the sterilizing device 14, with the loading surface 11 in front of the opening 15. The loading conveyor 41, with a load 60, is positioned in relation to the loading/unloading device 10 and a sensor G1 is triggered. Similarly, a sensor G2 is present in relation to the unloading surface 12. When triggered, i.e. when an object is detected, these sensors G1, G2 sends a blocking signal to the control unit, which is configured to control the positioning motor such that the housing 16 is not moved when the conveyor 41 is positioned in relation to the loading/unloading device 10.

In FIG. 7, the load has been moved from the loading conveyor 41 to the loading surface 11 (not shown). The loading surface 11 may also contain a sensor G3, which triggers a signal based on the load on the loading surface. Similarly, a sensor G4 may be provided onto the unloading surface 12. The control unit sends a cue signal to the sterilizing device 14 and if the sterilizing device 14 is ready to receive the load, the front door opening 151 is opened. When this is done, the sterilizing device 14 sends a signal to the loading/unloading device which triggers loading.

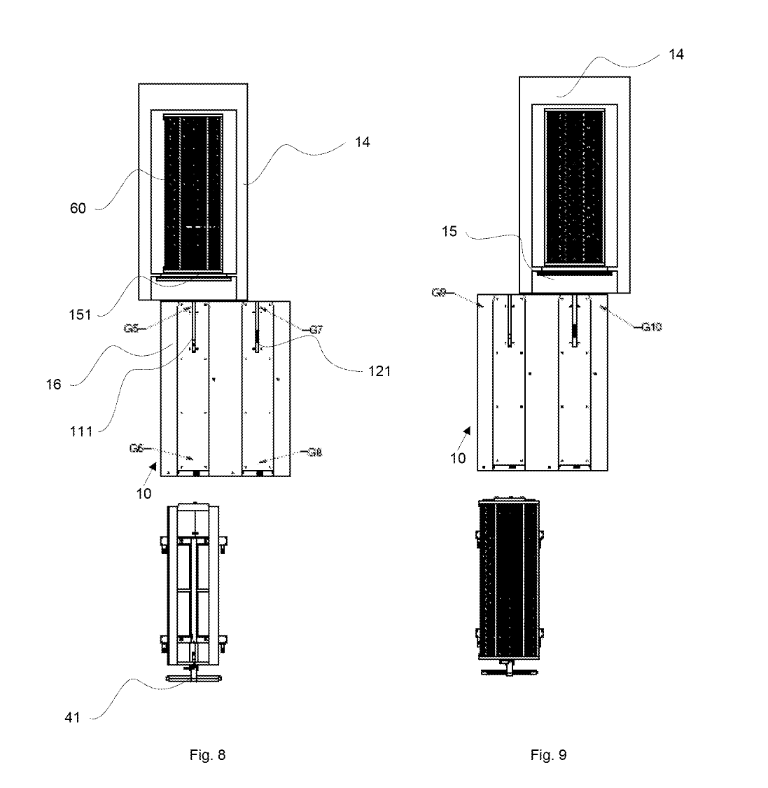

In FIG. 8, the load has entered the sterilizing device 14 and the loading arm 111 is in its extended state, triggering a sensor G5. This causes the loading means (not shown) to release the load and the arm to retract. When the arm is fully retracted, a sensor G6 is triggered. Analogously, the unloading arm 121 is correlated a sensor G7 and a sensor G8. The triggering of the sensor G6 causes the front door opening 151 to close and the sterilizing device 14 to start sterilization. It also causes the loading/unloading device 10 to shift position.

In FIG. 9, the position of the loading/unloading device 10 is shifted so that the unloading surface is in front of the opening 15. The shift occurs between a sensor G9 and a sensor G10, indicating the position of the loading/unloading device 10 in loading or unloading position, respectively. The loading/unloading device 10 may be guide steered sideways between the loading and unloading positions by means of an electrical motor as previously described. When the shift is completed, the loading/unloading device 10 sends a signal to the sterilizing device 14 that it is ready for unloading.

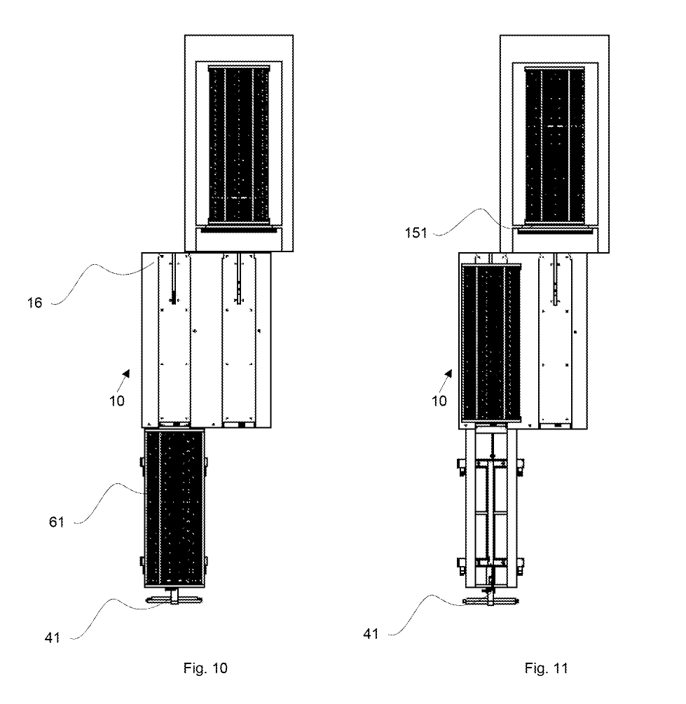

In FIG. 10, the loading/unloading device 10 is located in front of the sterilizing device 14, with the unloading surface 11 in front of the opening 15. Still, the loading conveyor 41, with a second load 61, is positioned in relation to the loading/unloading device 10 and the sensor G1 is triggered. This sends a blocking signal to the control unit, which is configured to control the positioning motor such that the housing 16 is not moved when the loading conveyor 41 is positioned in relation to the loading/unloading device 10.

FIG. 11 shows that the second load has been moved from the loading conveyor 41 to the loading surface 11 (not shown). The sensor G3 (not shown) is triggered by the load on the loading surface. The control unit sends a cue signal to the sterilizing device 14. When the sterilizing process is completed, and sensor G4 is not triggered, indicating that the loading/unloading device 10 is empty and thus ready to receive the load, the front door opening 151 is opened and the load is unloaded onto the unloading surface 12.

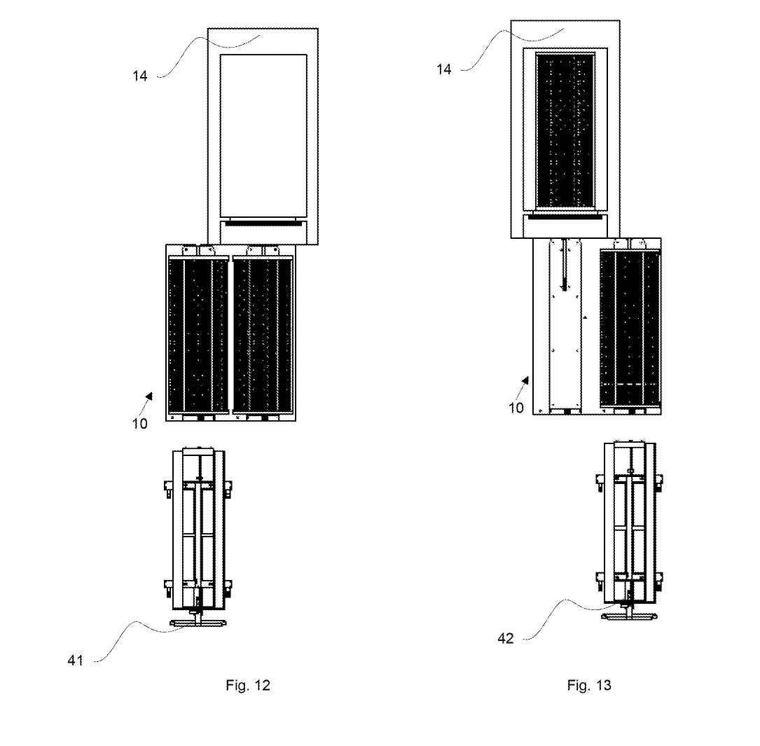

In FIG. 12, the loading conveyor 41 is removed from the loading/unloading device 10. Thus, since the seventh sensor is no longer triggered, it is possible for the loading/unloading device 10 to shift position between the sensor G9 (not shown) and the sensor G10 (not shown), as previously described.

In FIG. 13, since the control unit sends the cue signal to the sterilizing device 14, the sterilizing unit 14 is loaded with the second load and sterilization process starts. If the cue signal would not have been sent, indicating that no new load was present on the loading surface, the sterilizing device 14 may have entered standby-mode.

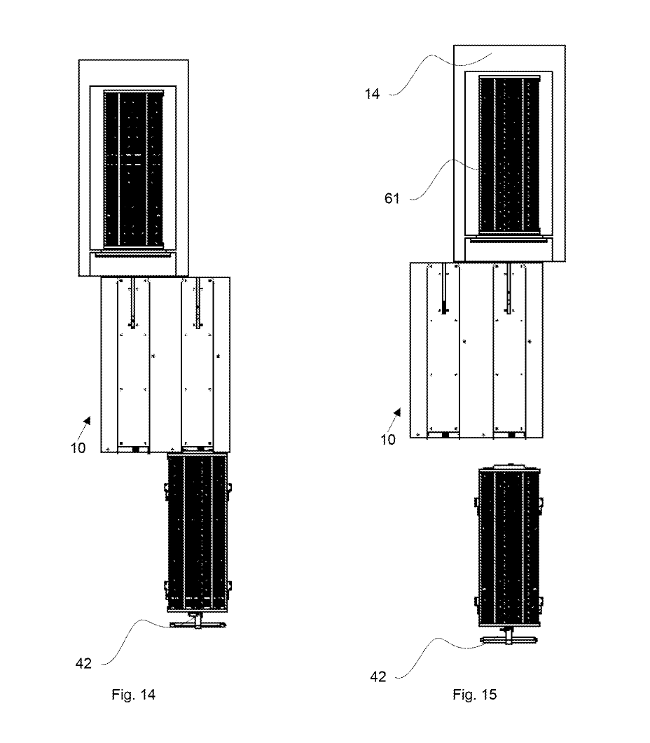

In FIG. 14, the load is unloaded onto the unload conveyor 42.

In FIG. 15, the unload conveyor 42 is removed from the loading/unloading device 10. Thus, since the sensor G2 is no longer triggered, it is possible for the loading/unloading device 10 to shift position between the sensor G9 (not shown) and the sensor G10 (not shown), as previously described. The loading/unloading device 10 is in unloading position, ready to receive the second load 61 from the sterilizing unit 14.

To facilitate for the personnel utilizing the loading/unloading device according to some embodiments, to avoid the possibility of loads to be sterilized of entering the unloading surface, or sterilized loads to enter the loading surface, the control unit is configured to control the lateral position of loading/unloading device such that the unloading surface may not be aligned with the loading conveyor, and the loading surface may not be aligned with the unloading conveyor.

The loading arm 111 is driven by a motor. The motor may drive the conveyor belt 11 in a forward direction, i.e. in accordance with FIG. 4 towards the sterilizing devices, such that a load may be collected from a loading conveyor and be transferred into the sterilizing device, as is shown in FIG. 4.

The unloading arm 121 is driven by a motor, optionally the same motor as is driving the loading arm 111. The motor may drive the unloading arm 12 in a backward direction, i.e. in accordance with FIG. 4 from the sterilizing devices, such that a load may be collected from the sterilizing device 14 and be transferred from the sterilizing device 14, optionally to the unloading conveyor 42.

FIG. 5 illustrates a sterilizing system 50 according to an embodiment, wherein in comparison to FIG. 4 a further sterilizing device 144 is provided between the loading conveyor 41, and the unloading conveyor 42. In this embodiment it is required that both the loading surface 11 and the unloading surface 12 may be used in both a forward and backward direction in order to be able to transport loads to and from all sterilizing devices comprised in the sterilizing system.

Thus, the loading surface 11 comprises a loading arm 111 and a second loading arm 511, which are slidably arranged in the loading surface, but in opposite directions. The loading arm 111 comprises an engaging loading means 112 as shown in FIG. 1a, such as a hook, for engaging the loading arm 111 to the load (not shown). Similarly, the second loading arm 511 comprises an engaging loading means, such as a hook, for engaging the second loading arm 511 to the load (not shown). When engaged the loading arm 111 is configured to move the engaged load into the sterilizing device 141, 142, 143 and the second loading arm 511 is configured to move the engaged load into the further sterilizing device 144.

The unloading surface 12 correspondingly comprises an unloading arm 121 and a second unloading arm 521, having an engaging unloading means 122 as shown in FIG. 1a, such as a hook, for engaging the loading arm 111 to a sterilized load (not shown) located in the sterilizing device 140, 142, 143. Similarly, the second unloading arm 521 comprises an engaging loading means, such as a hook, for engaging the second unloading arm 521 to the load (not shown). When engaged the unloading arm 121 is configured to move the engaged sterilized load from the sterilizing device 141, 142, 143 and onto the unloading surface 12 and the second unloading arm 521 is configured to move the engaged sterilized load from the further sterilizing device 144.

The loading arm 111 and second loading arm 511 and/or the unloading arm 121 and second unloading arm 521 with corresponding engaging loading and/or unloading means may be controlled by means of at least one motor (not shown) which is preferably comprised within the loading/unloading device 10.

The means for loading/unloading a load/sterilized load need not to be a loading arm or an unloading arm, as explained in the embodiments above. Instead, any conventional means, for loading and/or unloading the load/sterilized load to and from the loading surface/unloading surface, may be utilized without departing from the gist of the present invention. For example, the means for receiving a load may e.g. be a pair of wheels provided on the loading surface. The load may be transported onto, over, and from the loading surface by means of the wheels. The same solution may be provided on the unloading surface.

It should be appreciated that the invention is not limited to a certain type of means for laterally moving the device for alignment of the loading surface or the unloading surface to the sterilizing device and or loading or unloading conveyor. Any conventional means suitable for controllably, laterally moving the loading/unloading device in relation to the sterilizing device or loading or unloading conveyor may be utilized, without departing from the scope of the invention.

The control unit may be any unit normally used for performing the involved tasks, e.g. a hardware, such as a processor with a memory.

The sterilizing device according to the embodiments described above may e.g. be an autoclave. However, the present invention is not merely limited to this application. The sterilizing device could e.g. also be a dish washer or any other washing, cleaning or sterilizing device which benefits from the technical effects of the present invention.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A device for transferring a load into or from a sterilizing device, said device comprising:
a loading surface for transporting a load to be sterilized into an opening of the sterilizing device;
an unloading surface for transporting a sterilized load from the opening of the sterilizing device; and
a means for laterally moving the device in relation to the opening of the sterilizing device to align the loading surface or unloading surface with the opening of the sterilizing device.

2. The device according to claim 1, wherein the means comprises a rail along which the device be moved laterally to align the loading surface or unloading surface to the opening of the sterilizing device.

3. The device according to claim 2, comprising a corresponding recess for engaging the rail.

4. The device according to claim 1, wherein the loading surface comprises a slidably arranged loading arm arranged therein.

5. The device according to claim 1, wherein the unloading surface comprises a slidably arranged unloading arm arranged therein.

6. The device according to claim 1, further comprising control unit configured to control:
the lateral movement and alignment of the loading surface to the opening of the sterilizing device by receipt of a signal from a signal unit; and
the lateral movement and alignment of the unloading surface to the opening of the sterilizing device by receipt of a further signal sent form a signal unit.

7. The device according to claim 6 in combination with claim 4, wherein the control unit is configured to control the loading arm or unloading arm.

8. The device according to claim 6, wherein the signal unit is configured to send a signal to the control unit based on information from a sensor detecting at least one of the following:
a) when a new load is ready to be transferred onto the loading surface;
b) when a load has entered the loading surface;
c) when the sterilizing device is ready to receive a load;
d) when the door of the sterilizing device is opened;
e) when a load originally located on the loading surface has been transferred into the sterilizing device;
f) when an end of the loading surface or unloading surface is blocked by a load/sterilized load;
g) when the sterilization procedure of the sterilizing device is complete;
h) when the sterilized load has been transferred to the unloading surface; and
i) when the sterilized load has been transferred from the unloading surface.

9. The device according to claim 8, wherein the control unit by receipt of the signal is configured to control a positioning motor to align the loading surface with the opening of the sterilizing device based on information b), c), d),h), and i).

10. The device according to claim 8, wherein the control unit by receipt of the signal is configured to control a positioning motor to align the unloading surface with the opening of the sterilizing device based on information g).

11. The device according to claim 8, wherein the control unit by receipt of the signal is configured to control a positioning motor to align the loading surface with a loading conveyor based on information a), e), h), and i).

12. The device according to claim 8, wherein the control unit by receipt of the signal is configured to control a positioning motor to align the unloading surface with an unloading conveyor based on information h).

13. The device according to claim 8, wherein the control unit by receipt of the signal is configured to disable the positioning motor based on the information f).

14. The device according to claim 9, wherein the control unit is configured to control a loading arm to load a load positioned onto the loading surface into the sterilizing device, when the loading surface is aligned to the opening of the sterilizing device.

15. The device according to claim 11, wherein the control unit is configured to control a further loading arm to load a load positioned onto a loading conveyor onto the loading surface when the loading surface is aligned to the loading conveyor.

16. The device according to claim 10, wherein the control unit is configured to control an unloading arm to load a sterilized load from the sterilizing device onto the unloading surface, when the unloading surface is aligned to the opening of the sterilizing device.

17. The device according to claim 12, wherein the control unit is configured to control a further unloading arm to load a load positioned on the unloading surface onto an unloading conveyor when the unloading surface is aligned to the unloading conveyor.

18. The control unit according to claim 6.

19. A system comprising the device according to claim 1, and a sterilizing device.

20. The system according to claim 19, further comprising a loading conveyor and an unloading conveyor.

21. A method for loading a load into a sterilizing device comprising
receiving a load to be sterilized onto a loading surface of a device, the device further comprising an unloading surface;
aligning the loading surface with an opening of the sterilizing device, and
loading the load to be sterilized onto the sterilizing device.

22. A method for unloading a sterilized load from a sterilizing device comprising
aligning an unloading surface of a device further comprising a loading surface with an opening of the sterilizing device, and
unloading the sterilized load from the sterilizing device to the unloading surface of the device.
